(19) United States
(12) Patent Application Publication
(10) Pub. No.: US 2013/0052669 A1
(43) Pub. Date: Feb. 28, 2013

(54) COMPOSITIONS AND METHODS FOR RELIABLY DETECTING AND/OR MEASURING THE AMOUNT OF A MODIFIED TARGET PROTEIN IN A SAMPLE

(71) Applicants: Amanda G. Paulovich, Seattle, WA (US); Richard G. Ivey, Federal Way, WA (US)

(72) Inventors: Amanda G. Paulovich, Seattle, WA (US); Richard G. Ivey, Federal Way, WA (US)

(21) Appl. No.: 13/695,268

(22) PCT Filed: Apr. 29, 2011
(86) PCT No.: PCT/US2011/034659

§ 371 Date: Oct. 29, 2012

Related U.S. Application Data
(60) Provisional application No. 61/330,278, filed on Apr. 30, 2010.

(51) Int. Cl. G01N 33/566; G01N 33/53

(52) U.S. Cl. 435/7.92; 435/7.9; 436/501

(57) ABSTRACT

In one aspect, the invention provides a kit for detecting the presence or amount of a target modified protein within a sample. The kit comprises (i) a capture reagent that specifically binds to a first epitope on the target protein; (ii) at least one detection reagent that specifically binds to a second epitope on the target protein; and (iii) at least one synthetic hybrid reference peptide comprising the first epitope and the second epitope from the target protein of interest, wherein the synthetic reference peptide is capable of simultaneously binding to both the capture reagent and the at least one detection reagent, and wherein at least one of the first and second epitopes comprises a modified amino acid residue.

### CROSS-REFERENCE TO RELATED APPLICATION

This application claims the benefit of U.S. Patent Application No. 61/330,278, filed Apr. 30, 2010, the entire disclosure of which is hereby incorporated by reference herein.

### STATEMENT OF GOVERNMENT LICENSE RIGHTS

This invention was made with Government support under NIH U19 AI067770 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is: 36764_SEQ_FINAL.txt. The text file is 15 KB; was created on Apr. 29, 2011; and is being submitted via EFS-Web with the filing of the specification.

### FIELD OF THE INVENTION

The invention relates to methods, reagents and kits for detecting the presence and/or amount of a modified target protein in a sample.

### BACKGROUND

Protein quantification assays, such as ELISA assays require reference standards. Typically such reference standards are generated by cloning a protein of interest in an expression vector and producing and purifying recombinant protein. However, it is known that there are difficulties and expense associated with generating and purifying recombinant proteins, such as difficulties involved in cDNA cloning, optimization required for functional expression and purification of each protein, difficulties associated with purifying hydrophobic proteins, and the like. Moreover, a modified form of a recombinant protein, such as a phosphorylated form of a recombinant protein, is particularly challenging since it requires either enzymatic treatment of the recombinant protein or purification of the recombinant protein from a cell type that specifically modifies the protein in the desired manner.

Therefore, a need exists for reference standards that can be used to develop assays for detecting the presence and amount of proteins (including modified proteins) of interest, such as with an ELISA assay.

### SUMMARY

In one aspect, the invention provides a kit for detecting the presence or amount of a modified target protein within a sample. The kit comprises (i) a capture reagent that specifically binds to a first epitope on the target protein; (ii) at least one detection reagent that specifically binds to a second epitope on the target protein; and (iii) at least one synthetic hybrid reference peptide comprising the first epitope and the second epitope from the target protein of interest, wherein the synthetic reference peptide is capable of simultaneously binding to both the capture reagent and the at least one detection reagent, and wherein at least one of the first and second epitopes comprises a modified amino acid residue.

In another aspect, the invention provides a method for generating an assay for reliably detecting the presence or amount of a modified target protein within a sample. The method comprises (i) generating a capture reagent that specifically binds to a peptide corresponding to a first epitope on a target protein; (ii) generating at least one detection reagent that specifically binds to a peptide corresponding to a second epitope on the target protein; (iii) generating at least one synthetic hybrid reference peptide comprising the amino acid sequence of the first epitope and the amino acid sequence of the second epitope from the target protein of interest, wherein the synthetic reference peptide is capable of simultaneously binding to both the capture reagent and the at least one detection reagent, and wherein at least one of the first epitope and second epitope comprises a modified amino acid residue; and (iv) using the synthetic hybrid reference peptide as a quantitative standard for either (1) generating a standard curve for the assay, or (2) as a control in the assay.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates the structure of the human Smc1 protein and Smc1 peptide fragments used as immunogenic peptides and synthetic peptide standards, as described in Example 1.

FIG. 2A is a diagrammatic illustration of the use of a synthetic phosphopeptide reference molecule FHC37_FCp, derived from Smc1, in a sandwich ELISA assay format. In the illustrated embodiment, the detection mAb is a bivalent antibody that is conjugated with a detectable agent, indicated by the star symbol, which can be an agent such as a colloidal gold particle; as described in Example 1.

FIG. 2B illustrates the detection of a phosphorylated Smc1 polypeptide in a sandwich ELISA assay format, indicating the relative positions of capture mAb, the Smc1 protein, and detection mAb. In the illustrated embodiment, the detection mAb is a bivalent antibody that is biotinylated (B), and a detectable agent, indicated by the star symbol, is a labeled biotin binding agent, such as streptavidin; as described in Example 1.

FIG. 3 graphically illustrates a standard curve that was generated by plotting the concentration of the synthetic reference phosphopeptide (SEQ ID NO:5) versus OD450, a measure of the formation of a binding complex between the biotinylated detection mAb, the capture mAb, and the synthetic reference phosphopeptide, as described in Example 1.

FIG. 4 graphically illustrates the phospho-Smc1 (p966) concentration in Lymphoblast Cell Line (LBL) cells, wherein the LBL was divided and exposed to no or increasing doses of ionizing radiation (IR), lysed, and subjected to the phospho-Smc1 (p966) ELISA. The resulting OD450 value for each lysate was converted to a molar concentration by way of the equation of the line generated from the standard reference peptide (illustrated in FIG. 3), as described in Example 1.

### DETAILED DESCRIPTION

Unless specifically defined herein, all terms used herein have the same meaning as they would to one skilled in the art of the present invention.

As used herein, the term “modification” of a protein or peptide of interest includes any amino acid sequence whose chemical structure deviates from that found in the twenty naturally occurring amino acids and the peptide bonds that they form. Examples include one or more of the following acetylation, amidation, deamidation, prenylation, formylation, glycosylation, hydroxylation, methylation, myristoylation, phosphorylation, ubiquitination, ribosylation and/or sulphation, added to the protein post-translationally, co-translationally, or during a chemical synthesis. Additionally, proteins may be modified in vitro using chemical derivatizations, such as, for example, isotope-coded affinity tag (ICAT), and the like.

As used herein, the term “phosphorylation site” refers to an amino acid or amino acid sequence of a natural binding domain or a binding partner which is recognized by a kinase or phosphatase for the purpose of phosphorylation (e.g., phosphorylation on tyrosine, serine or threonine) or dephosphorylation of the polypeptide or a portion thereof.

As used herein, the term “affinity reagent” refers to any molecule that has affinity for binding to the target molecule of interest. As used herein, affinity reagent includes one or more of the following: a) aptamers; b) affinity reagents identified through screening phage display, chemical, or yeast libraries; c) any of the classes of immunoglobulin molecules of any species, or any molecules derived therefrom, including whole antibodies and any antigen-binding fragment (i.e., “antigen-binding portion”) or single chains thereof. Exemplary antibodies include polyclonal, monoclonal, single chain, and recombinant antibodies. The terms “monoclonal antibody” or “monoclonal antibody composition” as used herein refer to a preparation of antibody molecules of single molecular composition. A monoclonal antibody composition displays a single binding specificity and affinity for a particular epitope.

As used herein, the term “anti-peptide affinity reagent” refers to any type of affinity reagent (in the preceding general sense) that binds to a peptide for the purpose of enrichment and/or detection of a polypeptide comprising the peptide from a biological sample or processed sample. As used herein, an affinity reagent that “specifically binds to a target peptide” is intended to refer to an affinity reagent that binds to the peptide with a K_{D} of 1×10⁻³M or less.

The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for an affinity reagent can be determined using methods well established in the art. An exemplary method for determining the K_{D} of an affinity reagent is by using surface plasmon resonance, or using a biosensor system such as a Biacore® system.

As used herein, the term “epitope” refers to the precise chemical structures of a peptide or protein (or modified versions thereof) that interact with an affinity reagent.

As used herein, the terms “immunogen” and “antigen” refer to the peptide or protein (or modified versions thereof) to which an affinity reagent was generated.

As used herein, the term “affinity” refers to the strength of interaction between an affinity reagent and antigen at their interaction sites. Within each interaction site, the affinity reagent interacts through chemical forces with the target at numerous sites; the more interactions, the stronger the affinity.

As used herein, the term “cross-reactivity” refers to an affinity reagent or population of affinity reagents binding to epitopes on other antigens. This can be caused either by imperfect specificity of the affinity reagent or by multiple distinct antigens having identical or very similar epitopes. Cross reactivity is sometimes desirable when one wants general binding to a related group of antigens or when attempting cross-species labeling when the antigen epitope sequence is not highly conserved in evolution.

As used herein, the term “about” refers to plus or minus ten percent (10%) of the referenced value.

The present invention is based, at least in part, on the discovery by the present inventors that a synthetic hybrid peptide reference standard comprising at least two separate epitopes (i.e., one for capture and another for detection of the protein or peptide of interest) can be generated for any protein of interest, thereby avoiding the need for cloning, expressing, purifying and phosphorylating, or otherwise modifying a recombinant protein for use as a reference standard in a quantitative assay. The methods and reagents of the invention can be used to assess the status of any of one or more modification(s) of a protein of interest, such as, for example, protein phosphorylation, present in a biological sample of interest, such as a biological fluid or a biological tissue or cell line. Additionally, the methods and reagents of the invention can be used to assess the concentration of any unmodified protein of interest, present in a biological sample of interest, such as a biological fluid or a biological tissue. Examples of biological fluids include urine, blood, plasma, serum, saliva, semen, stool, sputum, cerebral spinal fluid, tears, mucus, amniotic fluid or the like. Examples of biological tissues include organs, tumors, lymph nodes, arteries and individual cells. The sample may also be a mixture of target protein containing molecules prepared in vitro. The methods, reagents and kits of the invention provide the ability to generate a quantitative assay, such as an ELISA assay for any protein of interest, without the limitations of generating a recombinant protein for a reference standard. Also, the hybrid peptide reference standards can be synthesized in large amounts at high purity. Batch to batch variation can be assessed by mass spectroscopy and quantified by amino acid analysis.

In accordance with the foregoing, in one aspect, a method is provided for generating an assay for reliably detecting the presence or amount of a modified or unmodified target protein within a sample. The method comprises (i) generating a capture affinity reagent that specifically binds to a peptide corresponding to a first epitope on a target protein; (ii) generating at least one detection affinity reagent that specifically binds to a peptide corresponding to a second epitope on the target protein, (iii) generating at least one synthetic reference hybrid peptide comprising the amino acid sequence of the first epitope and the amino acid sequence of the second epitope from the target protein of interest, wherein the synthetic hybrid reference peptide is capable of simultaneously binding to both the capture reagent and the at least one detection reagent; and (iv) using the synthetic hybrid reference peptide as a quantitative standard for either (1) generating a standard curve for the assay, or (2) as a control in the assay.

Selection of the First Epitope for Binding to a Capture Reagent

The first epitope is selected for binding to an affinity reagent, such as a capture antibody and corresponds to the protein of interest and serves as a unique recognition sequence for binding to a capture reagent (e.g., with at least detectable selectivity). When referring herein to “uniqueness” with respect to the first epitope and/or the second epitope, the epitope may be an amino acid sequence that is truly unique to the protein from which it is derived. Alternatively, it may be unique just to the sample from which it is derived (e.g., human sample), but the same amino acid sequence may be present in, for example, the murine genome. Alternatively, the epitope may be an amino acid sequence that is not unique to one protein, but rather is a motif present in a family of proteins. Epitopes in a protein of interest may be identified using Web-based tools to predict antigenic peptide, such as, for example, the method of Kolaskar AS and Tongaonkar PC, FEBS Lett 276:172-4 (1990).

The first epitope may be determined in silico and generated in vitro, such as by peptide synthesis, without cloning or purifying the protein it derives from. The first epitope for binding may be selected by performing a comprehensive search of one or more relevant databases using all theoretically possible epitopes of the protein of interest with a given length (e.g., from 5 to about 150 continuous amino acid residues in length from a protein of interest). This process is preferably carried out computationally using any of the sequence search tools available in the art. For example, to identify a first epitope from a protein of interest having at least 5 continuous amino acid residues in length, immunogenic domains in the protein of interest may be identified using Web-based tools to predict antigenic peptide, such as, for example, the method of Kolaskar AS and Tongaonkar PC, FEBS Lett 276:172-4 (1990). In some embodiments, the first epitope for binding to a capture reagent is from 5 amino acids in length up to about 150 amino acids in length, such as from 5 to about 25 amino acids in length, such as from 5 to about 75 amino acids in length.

The first epitope may be derived from any portion of the full length protein of interest. In some embodiments, the first epitope is derived from the amino half of the protein of interest (i.e., an amino acid sequence 5′ of the mid point of the protein coding sequence). In some embodiments, the first epitope is derived from the carboxy half of the protein (i.e. an amino acid sequence 3′ of the mid point of the protein coding sequence).

A synthetic peptide comprising the amino acid sequence of the first epitope may be used to raise a capture affinity reagent, such as an antibody specific for the first epitope, as described in Example 1.

Selection of the Second Epitope for Binding to a Detection Affinity Reagent

The second epitope is selected for binding to a detection reagent, such as a detection antibody that selectively binds to a post-translationally modified (e.g., phosphorylated) form of the protein at a specific site of interest. The second epitope is selected to correspond to the protein of interest and serves as a recognition sequence for binding to a detection reagent (e.g., with at least detectable selectivity).

In some embodiments, the second epitope is an amino acid sequence comprising from 5 to about 150 amino acid residues of the target protein of interest (such as from 5 to about 25 amino acids in length, such as from 5 to about 75 amino acids in length).

A synthetic peptide comprising the amino acid sequence of the second epitope may contain a modified site, such as a phosphorylated serine, tyrosine or threonine, or others such as a modified acetylation, amidation, deamidation, prenylation, formylation, glycosylation, hydroxylation, methylation, myristoylation, phosphorylation, ubiquitination, ribosylation and/or sulphation site, or a site on a protein modified in vitro using a chemical derivitization (e.g. ICAT), to be detected, which may be used to raise a detection agent, such as an anti-phospho antibody, or other affinity reagents specific to the modified site, as described in Example 1.

The invention also encompasses the selection and simultaneous use of multiple detection epitopes for binding to multiple detection affinity reagents. Therefore, in addition to a capture reagent, the target peptide and the synthetic hybrid reference peptides can comprise two, three or more detection epitopes. In these embodiments, the capture reagent and the detection reagents for each epitope preferably all are capable of simultaneously binding to the target peptide or synthetic hybrid reference peptide. At least one of the plurality of epitopes contains a modified amino acid residue. In some embodiments, two, three, or all of the epitopes contain a modified amino acid residue.

Generation of a Synthetic Hybrid Reference Peptide for Use as a Quantitation Standard

The method further comprises generating a synthetic hybrid reference peptide comprising the first epitope and the second epitope from the target protein of interest, wherein the synthetic hybrid reference peptide is capable of simultaneously binding to both the capture reagent and the at least one detection reagent.

In some embodiments, the synthetic hybrid reference peptide is a phosphopeptide comprising a phosphorylated amino acid at a post-translational modification site of interest.

In some embodiments, the synthetic hybrid reference peptide further comprises an amino acid spacer region from 1 to about 50 amino acid residues between the first and second epitopes. In some embodiments, a potential post-translational modification site is positioned in the synthetic hybrid reference peptide such that at least 1 to 10 amino acid residues separate the first epitope from the potential post-translational modification site.

In embodiments wherein the target peptide contains two, three or more detection epitopes for binding to detection affinity reagents, the reference hybrid standard peptide can be generated to also contain the two, three or more detection epitopes, in addition to the capture epitope. Therefore, in some embodiments the synthetic hybrid reference peptide further comprises at least one amino acid spacer region from 1 to about 50 amino acid residues between. The amino acid spacer region can be disposed between any two of the epitopes. For example, in a synthetic hybrid standard peptide with one capture epitope and two detection epitopes, the synthetic hybrid standard peptide can contain two amino acid spacer regions, one disposed between each of the neighboring pairs of epitopes.

Generation of Capture Affinity Reagents

As used herein, the term “capture affinity reagent’ includes any affinity reagent which is capable of binding to a target protein that includes the first epitope, with at least detectable selectivity. In a preferred embodiment, the capture reagent is an antibody or a fragment thereof, such as a polyclonal antibody, or a monoclonal antibody or fragment thereof, or a single chain antibody or a reagent selected from a displayed library.

In accordance with the methods of the invention, a capture reagent is generated that binds to a first epitope on a protein of interest. Any art-recognized method can be used to generate a capture reagent that specifically binds to the first epitope. For example, a synthetic immunopeptide comprising the first epitope can be generated, either with or without an N-terminal spacer sequence, for example, as described in Example 1. The immunopeptide can be used alone or linked to an immunostimulatory agent and used to immunize a suitable subject (e.g., rabbit, goat, mouse, or other mammal or vertebrate) or to screen a display library (e.g., phage, yeast, aptamer). If a subject is immunized, at the appropriate time after immunization, antibody-producing cells can be obtained from the subject and used to prepare monoclonal antibodies by standard techniques, such as the hybridoma technique originally described by Köhler, G., and C. Milstein, “Continuous Cultures of Fused Cells Secreting Antibody of Predefined Specificity,” Nature 256(5517):495-7, Aug. 7, 1975, incorporated herein by reference. Once the candidate capture agent antibodies are generated, the candidate antibodies may be screened for affinity to the target protein to identify the most suitable antibodies for use as a capture reagent.

A plurality of capture reagents may be attached to a support having a plurality of discrete regions (features), such as an array or test strip. The capture reagent array can be produced on any suitable solid surface, including silicon, plastic, glass, polymer, such as cellulose, polyacrylamide, nylon, polystyrene, polyvinyl chloride or polypropylene, ceramic, photoresist or rubber surface. Preferably, the silicon surface is a silicon dioxide or a silicon nitride surface. Also preferably, the array is made in a chip format. The solid surfaces may be in the form of tubes, beads, discs, silicon chips, microplates, polyvinylidene difluoride (PVDF) membrane, nitrocellulose membrane, nylon membrane, other porous membrane, non-porous membrane, e.g., plastic, polymer, perspex, silicon, amongst others, a plurality of polymeric pins, or a plurality of microtitre wells, or any other surface suitable for immobilizing proteins and/or conducting an immunoassay or other binding assay.

Generation of Detection Affinity Reagents

A detection affinity reagent, such as an antibody, is generated that specifically binds to a second epitope on the protein of interest, which in some embodiments may contain a modification of interest, such as a phosphorylation site, whereas in other embodiments, it may not contain modification. In some embodiments, the detection antibody is specific for said post-translational modification. In some embodiments, the detection antibody is an anti-phospho antibody that specifically binds to a phosphorylated site in the second epitope derived from the protein of interest.

Any art-recognized method can be used to generate a detection reagent that specifically binds to the second epitope, either in the modified or unmodified form. For example, a synthetic immunopeptide comprising the second epitope can be generated, either with or without an N-terminal spacer sequence, for example as described in Example 1. The immunopeptide can be used alone or linked to an immunostimulatory agent and used to immunize a suitable subject (e.g., rabbit, goat, mouse, or other mammal or vertebrate), or to screen a display library (e.g., phase, yeast, aptamer). If a subject is immunized, at the appropriate time after immunization, antibody-producing cells can be obtained from the subject and used to prepare monoclonal antibodies by standard techniques, such as the hybridoma technique originally described by Köhler and Milstein (1975). Once the candidate detection antibodies are generated, the candidate antibodies may be screened for affinity to the target protein to identify the most suitable antibodies for use as a detection reagent.

In some embodiments, the detection agent, such as an anti-phospho antibody, is labeled with a detectable moiety such as an enzyme, a fluorescent label, a stainable dye, a chemilumninescent compound, a colloidal particle, a radioactive isotope, a near-infrared dye, a DNA dendrimer, a water-soluble quantum dot, a latex bead, a selenium particle, or a europium nanoparticle.

In one embodiment, post-translational modification is phosphorylation, and said detection reagent is a labeled antibody specific for phosphorylated tyrosine, phosphorylated serine, or phosphorylated threonine. In one embodiment, said detection antibody is labeled by an enzyme or a fluorescent group. In one embodiment, said enzyme is HRP (horse radish peroxidase). In one embodiment, said post-translational modification is phosphorylation, and said detection reagent is labeled with a fluorescent dye that specifically stains phosphoamino acids. In one embodiment, said fluorescent dye is Pro-Q Diamond dye. In one embodiment, the detection reagent is labeled with biotin, wherein colorimetric detection is indicative of binding to an avidin-HRP conjugate.

In some embodiments, Enzyme-Linked Immunosorbent Assay (ELISA) is used for detection of a protein that interacts with a capture reagent. In an ELISA, the indicator molecule is covalently coupled to an enzyme and may be quantified by determining with a spectrophotometer the initial rate at which the enzyme converts a clear substrate to a correlated product. Methods for performing ELISA are well known in the art and described in, for example, Perlmann, H., and P. Perlmann, “Enzyme-Linked Immunosorbent Assay,” Cell Biology: A Laboratory Handbook, Academic Press, Inc., San Diego 1994, pp. 322-328; Crowther, J. R., Methods in Molecular Biology, Vol. 42-ELISA: Theory and Practice, Humana Press, Totowa, N.J., 1995; Harlow, E., and D. Lane, “Antibodies: A Laboratory Manual,” Cold Spring Harbor Laboratory Press, Cold Spring Harbor, N.Y., pp. 553-612, 1988, the contents of each of which are incorporated by reference. Sandwich (capture) ELISA may also be used to detect a protein that interacts with a capture affinity reagent (e.g., capture antibody) and a detection affinity reagent (e.g., detection antibody). Sandwich ELISAs for the quantitation of proteins of interest are especially valuable when the concentration of the protein in the sample is low and/or the protein of interest is present in a sample that contains high concentrations of contaminating proteins.

Preparation of Synthetic Hybrid Reference Peptides:

Synthetic hybrid reference peptides can be prepared by classical methods known in the art, for example, by using standard solid phase techniques. The standard methods include exclusive solid phase synthesis, partial solid phase synthesis methods, fragment condensation, and classical solution synthesis. Solid phase peptide synthesis procedures are well known in the art and further described by John Morrow Stewart, Solid Phase Peptide Synthesis (2nd Ed. Pierce Chemical Company, 1984). Synthetic peptides can be purified by preparative high performance liquid chromatography and the composition of which can be confirmed via amino acid sequencing. Once the capture affinity reagent and the detection affinity reagent are available, a standard curve is generated using the hybrid reference peptide standard, and the concentration of the target protein in any given sample can be readily determined in an assay using the reagents described herein, for example in an ELISA assay.

For example, as described in Example 1, ELISA assays were developed to detect phosphorylation in the target protein Smc1 using the methods described herein. While Example 1 describes detection of serine phosphorylation in the Smc1 protein, it will be understood by those of skill in the art that the methods described herein may be applied to detect and/or quantify any protein of interest, modified or not, to which affinity reagents can be generated. Generation of an ELISA assay is well known in the art and requires the following parameters: (1) binding of a capture antibody to a solid surface support (e.g., a 96 well plate); (2) a biospecimen; (3) a quantification standard; (4) a labeled detection antibody (e.g., biotinylated); and (4) detection reagents, such as an enzyme-avidin conjugated reagent and a colorimetric substrate.

In another aspect of the invention, a kit is provided for detecting the presence or amount of a target protein within a sample. The kit comprises (i) a capture reagent that specifically binds to a first epitope on the target protein; (ii) at least one detection reagent that specifically binds to a second epitope on the target protein; and (iii) at least one synthetic hybrid reference peptide comprising the first epitope and the second epitope from the target protein of interest, wherein the synthetic reference peptide is capable of simultaneously binding to both the capture reagent and the at least one detection reagent. The capture reagents, detection reagents and synthetic hybrid reference peptides may be generated as described herein.

In some embodiments, the kit further comprises reagents for conducting an immunoassay, such as an ELISA assay. If lateral flow test strips are to be used to conduct the immunoassay, then the antibodies within the kit, and optionally the synthetic reference standards, will be embedded in the lateral flow test trips.

While various embodiments of the invention herein are described in the context of a capture affinity reagent binding to a first epitope on a protein of interest and a detection affinity reagent binding to a second epitope (such as a modified site) on the protein of interest, the invention is not intended to be so limited. It will be understood by those of skill in the art that a protein quantification assay in accordance with the claimed invention can also be carried out in reverse, for example a phospho-protein may be captured by an anti-phospho antibody and detected with an antibody that specifically binds to any other epitope on the protein, and such embodiments are intended to be encompassed by the present invention.

The following examples merely illustrate the best mode now contemplated for practicing the invention, but should not be construed to limit the invention.

### Example 1

This Example describes the generation of monoclonal antibodies against phosphorylated forms of Structural Maintenance of Chromosomes 1 (“Smc1”) (Smc1 pS957 and pS966), the generation of synthetic hybrid reference phosphopeptides, and the development of ELISA assays for use therewith.

Rationale:

It was determined that phospho-Smc1S957 and phospho-Smc1S966 are induced in cells exposed to radiation, and in dogs exposed to total body irradiation. This Example describes the generation of reagents for measuring the presence and/or amount of phospho-Smc1 (pS957) and/or phospho-Smc1 (pS966) in a biological sample, including the use of synthetic hybrid reference phosphopeptide comprising the first epitope (capture) and either the second epitope (pS957) of interest or the third epitope of interest (pS966) from the Smc1 target protein, with reference to the human full length sequence, included here in as SEQ ID NO:6, wherein the synthetic hybrid reference peptide is capable of simultaneously binding to both the capture reagent that binds to the first epitope and to at least one detection reagent that binds to the second or third epitope. While the Example is described with reference to the Smc1 protein, it will be understood by those of skill in the art that the methods described may be applied to generate reagents and methods for reliably detecting and/or measuring the amount of any target protein, modified or not, to which affinity reagents can be generated.

1. Selection of Epitope #1 from Smc1 for Binding to a Capture Agent

The first epitope was derived from the carboxyl-terminus of the Smc1 protein, which is highly conserved between human, monkey, rabbit, dog, mouse and rat, as shown below in Table 1. “Reference ID” refers to the amino acid sequence of the full length Smc1 protein.

TABLE 1
Smc1: Alignment of conserved Regions corresponding to Epitope 1
(Capture epitope)

| | | Region corresponding to FHC37F synthetic |
|---|---|---|
| organism | reference ID | peptide (capture epitope = epitope #1) |
| synthetic | FHC37F | DLTKYPDANPNPNEQ (SEQ ID NO: 1) |
| peptide | | |
| human | NP_006297.2 | DLTKYPDANPNPNEQ (SEQ ID NO: 1) |
| monkey | XP_001091228.1 | DLTKYPDANPNPNEQ (SEQ ID NO: 1) |
| rabbit | XP_002720089.1 | DLTKYPDANPNPNEQ (SEQ ID NO: 1) |
| dog | XP_538049.2 | DLTKYPDANPNPNEQ (SEQ ID NO: 1) |
| mouse | NP_062684.2 | DLTKYPDANPNPNEQ (SEQ ID NO: 1) |
| rat | NP_113871.1 | DLTKYPDANPNPNEQ (SEQ ID NO: 1) |

2. Selection of Epitope #2 for Detection of Phosphorylated Smc1 at Serine 957

An amino acid region corresponding to the second epitope of Smc1 was selected (SQEEGS[p]SQGEDSVSG included herein as SEQ ID NO:2) which corresponds to human Smc1 amino acids 951 to 965 and was synthesized with a phospho-serine at position 957, with reference to the human full length sequence, included here in as SEQ ID NO:6.

TABLE 2
Smc1: Alignment of conserved Regions corresponding to Epitope #2
(Detection of phosphorylated Serine 957 (p957))

| | | Region corresponding to FHC37Cp |
|---|---|---|
| | | synthetic peptide (detection epitope = |
| organism | reference ID | epitope #2) |
| synthetic | FHC37Cp | SQEEGSSQGEDS (SEQ ID NO: 14) |
| peptide | | |
| human | NP_006297.2 | SQEEGSSQGEDS (SEQ ID NO: 14) |
| monkey | XP_001091228.1 | SQEEGSSQGEDS (SEQ ID NO: 14) |
| rabbit | XP_002720089.1 | SQEEGSSQGEDS (SEQ ID NO: 14) |
| dog | XP_538049.2 | SQEEGSSQGEDS (SEQ ID NO: 14) |
| mouse | NP_062684.2 | SQEEGSSQGE [symbol] S (SEQ ID NO: 7) |
| rat | NP_113871.1 | SQEEG [symbol] SQGE [symbol] S (SEQ ID NO: 8) |

Note:
The sequence set forth as SEQ ID NO: 14 is fully contained within the sequence set forth in SEQ ID NO: 2.

3. Selection of Epitope #3 for Detection of Phosphorylated Smc1 at Serine 966

An amino acid region corresponding to the third epitope of Smc1 was selected (DSVSG[p]SQRISS, included herein as SEQ ID NO:3) which corresponds to human Smc1 amino acids 961 to 971 with a phospho-serine at protein position 966, with reference to the human full length sequence, included here in as SEQ ID NO:6.

TABLE 3
Smc1: Alignment of conserved Regions corresponding to Epitope #3
(Detection of phosphorylated Serine 966 (p966))

| | | Region corresponding to FHC37Dp |
|---|---|---|
| | | synthetic peptide (detection epitope = |
| organism | reference ID | epitope #3) |
| synthetic | FHC37Dp | DSVSGSQRISS (SEQ ID NO: 3) |
| peptide | | |
| human | NP_006297.2 | DSVSGSQRISS (SEQ ID NO: 3) |
| monkey | XP_001091228.1 | DSVSGSQRISS (SEQ ID NO: 3) |
| rabbit | XP_002720089.1 | DSVSGSQR [symbol] SS (SEQ ID NO: 9) |
| dog | XP_538049.2 | DSVSGSQR [symbol] SS (SEQ ID NO: 9) |
| mouse | NP_062684.2 | [symbol] SVSGSQR [symbol] SS (SEQ ID NO: 10) |
| rat | NP_113871.1 | [symbol] SVSGSQR [symbol] SS (SEQ ID NO: 10) |

4. Generation of Anti-Phospho-Smc1 Antibodies that Bind to Epitope #2 (pS957) or Epitope #3 (p966) Using Synthetic Immunogenic Peptides

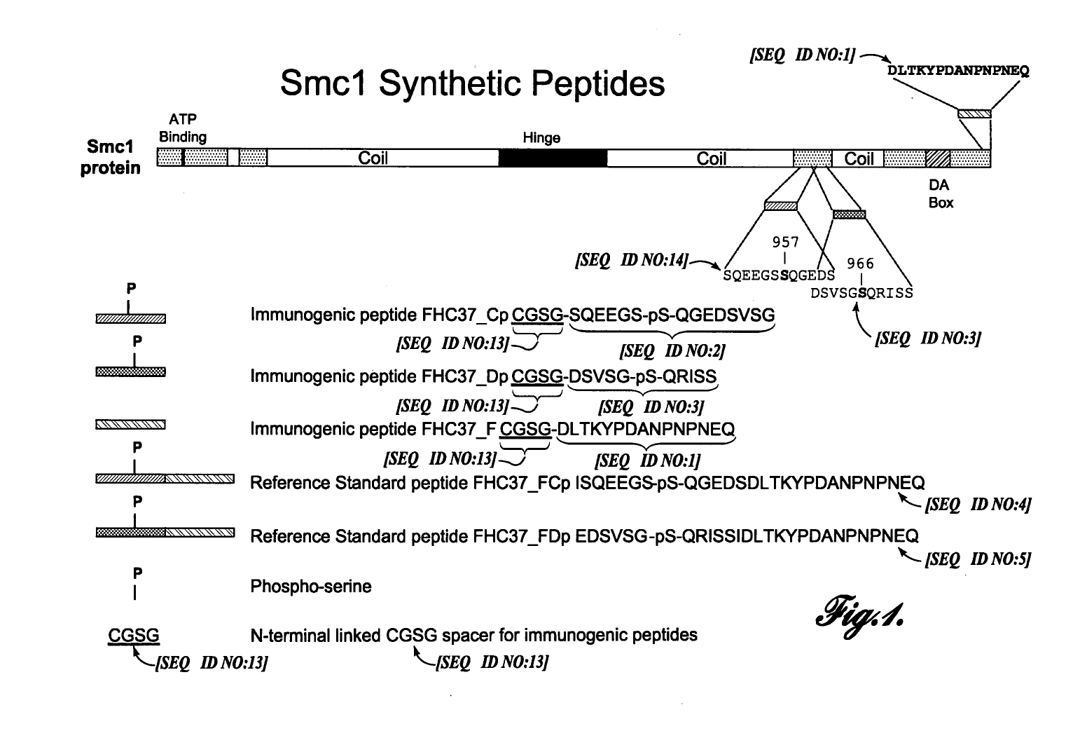

Monoclonal antibodies were generated against the serine phosphorylated forms of Smc1 as follows. Phosphorylated peptides encompassing the pS957 or pS966 amino acids of Smc1 and a peptide (not phosphorylated) corresponding to the capture epitope were synthesized as shown in Table 4 and the diagram illustrated in FIG. 1.

TABLE 4
Synthetic Immunogenic Peptides and Synthetic Reference
phospho-peptides

| | | SEQ ID |
|---|---|---|
| Description | Sequence | NO: |
| Immunogenic peptide FHC37_F | DLTKYPDANPNPNEQ | 1 |
| (capture) (N term CGSG spacer was | | |
| added) | | |
| Immunogenic phospho-peptide | SQEEGS[p]SQGEDSVSG | 2 |
| FHC37_Cp/Serine 957 (detection) | | |
| (N term CGSG spacer was added) | | |
| Immunogenic phospho-peptide | DSVSG[p]SQRISS | 3 |
| FHC37_Dp/Serine 966 (detection) | | |
| (N term CGSG spacer was added) | | |
| synthetic peptide reference standard | ISQEEGS[p]SQGEDSDLTKYPD | 4 |
| FHC37_FCp | ANPNPNEQ | |
| | (phosphorylated Serine 957) | |
| synthetic peptide reference standard | EDSVSG[p]SQRISSIDLTKYPDA | 5 |
| FHC37_FDp | NPNPNEQ | |
| | (phosphorylated Serine 966) | |

Note:
[p]S: designates a phosphorylated serine residue

All peptides used for immunization, screening and standards were synthesized by Chinese Peptide Company (Hangzhou, China). Three peptides were used for immunization; the first peptide (SQEEGS[p]SQGEDSVSG) corresponds to human Smc1 amino acids 951 to 965, as included herein as SEQ ID NO:2, and was synthesized with a phospho-serine at position 957. The second immunization peptide (DSVSG[p]SQRISS) corresponds to human Smc1 amino acids 961 to 971, as included herein as SEQ ID NO:3, with a phospho-serine at protein position 966. The third immunization peptide (DLTKYPDANPNPNEQ) corresponds to the C-terminus of the Smc1 protein starting at position 1219, as included herein as SEQ ID NO:1. All three immunization peptides were synthesized with an N-terminal linked CGSG spacer, included herein as SEQ ID NO:13.

Two peptides were synthesized for counter screening and are the non-phosphorylated counterparts of the two phospho immunization peptide: (CGSGSQEEGSSQGEDS and CGSGDSVSGSQRISS, included herein as SEQ ID NOS:11 and 12, respectively). Two additional peptides were generated for reference standards (ISQEEGS-pS-QGEDSDLTKYPDANPNPNEQ, SEQ ID NO:4, and EDSVSG-pS-QRISSIDLTKYPDANPNPNEQ, SEQ ID NO:5). Both the reference standard peptides contain the C-terminal sequence of Smc1 (AAs 1219 to 1233) concatenated with the sequence surrounding pS957 or pS966. Standard peptide concentrations were determined by Amino Acid Analysis (New England Peptide, Gardner, Mass.).

The synthesized immunopeptides were conjugated to Keyhole Limpet Hemocyanin (KLH) and used to immunize 12 rabbits at a commercial facility (Epitomics, Burlingame, Calif.). The rabbits were bled prior to immunization and then injected with the KLH-conjugated Smc1 peptides and boosted every 2-3 weeks for a total of 5-6 injections per rabbit. The rabbits were monitored for immune response by peptide ELISA, and were also counter-screened with the corresponding non-phosphorylated peptide. The rabbits were scored as passing the peptide ELISA screen based on empirical criteria (O.D.>0.30 for the 1:64,000 serum dilution).

Final sera from immunized rabbits were screened by Western Blot and Immunoprecipitation. Regarding the Western Blot (WB) analyses, whole cell lysates were isolated from human LBL at either 2 or 5 hours after treatment with mock or 10 Gy of IR (5.6 Gy/min). Lysates were subjected to SDS-PAGE, transferred to nitrocellulose membranes and probed with Protein-A purified antibody isolated from pre- or post-immune rabbit sera. Positive controls were run by blotting the same lysates with a commercial antibody that binds the phosphorylated forms of SCM1. Additionally, the specificity of anti-phospho antibodies were confirmed by including a control lysate from cells treated with 10 Gy of IR followed by treatment with λ-phosphatase. An immunized rabbit was scored as positive by WB screen if the post-immune sera gave a signal at the appropriate molecular weight and the signal was absent from the corresponding pre-immune Western Blot.

Regarding Immunoprecipitation (IP) analyses, pre- or post-immune antibody was purified from sera by Protein-A affinity columns. The IP complex was brought down with either Protein-A or Protein-G agarose beads, reduced with 10 mM DDT, heat-denatured and subjected to SDS-PAGE. Proteins were transferred to nitrocellulose membranes, blocked, and then probed with an antibody directed toward the target protein of interest. A rabbit was scored as positive by Immunoprecipitation if the post-immune sera gave a signal at the appropriate molecular weight and the signal was absent from the corresponding pre-immune WB. Additionally, phospho-specificity of the antibodies was established if they resulted in an enriched signal in the 10 Gy lysate relative to the mock irradiated and the λ-phosphatase treated lysate.

Based on the screening data, three rabbits were selected for monoclonal antibody (mAb) production. Specifically, one (1) rabbit was selected for the generation of the pan (capture) mAb (i.e., capture agent that specifically binds to epitope #1), and the remaining two rabbits were selected for the generation of the two different phospho-specific mAbs (i.e., detection agents that bind to epitope #2 or epitope #3).

Primary hybridoma lines from the selected immunized rabbits were created and screened. Briefly, lymphocytes were isolated from the spleens of selected animals and fused to create rabbit hybridoma lines grown in multi-well plates that contained 1-5 clones per well. The goal of this step in the process was to identify the 3 best candidate hybridoma lines (and up to 3 backup lines) to be sub-cloned to generate monoclonal hybridoma lines. The supernatants from these multi-clone hybridoma lines were screened by a combination of peptide ELISA, IP, and Western Blot. A subset of the primary hybridoma lines were subcloned by serial dilution. Supernatants from subclones were screened by peptide ELISA, IP, and Western Blot to identify hybridoma cell lines generating the correct mAb.

ELISA assays were developed using the mAbs specific for Smc1, including pan (capture) mAb (i.e., capture agent that specifically binds to epitope #1), and the two different phospho-specific mAbs (i.e. detection agents that bind to epitope #2 or epitope #3). Multiple parameters were optimized in an iterative process in which two parameters at a time were compared before moving on to the next set of parameters. This process was repeated multiple times until the overall assay was optimized. For example, the initial parameter optimized was the concentration of capture Ab. Using high concentrations of sample (cell lysate or synthetic peptide), the amount of capture Ab per well was varied. A plot of the antibody concentration versus the signal for each sample concentration revealed that mAb concentration becomes limiting between 200 and 300 ng/well (not shown). Subsequently, the optimal dilution of biotinylated detection Ab was determined to be 1:4,000 by plotting the signal to noise ratio we detect an optimal detection Ab concentration for each batch of labeled detection Ab (not shown). Other parameters optimized along these lines include: ELISA plate composition, capture Ab binding buffer, blocking buffer composition, cell lysate buffer, sample concentration, sample incubation time, sample incubation temperature, standard curve dynamic range, detection Ab labeling method, HRP enzyme conjugate, different TMB substrate sources, and substrate development times. The key parameters affecting assay sensitivity were capture Ab concentration, detection Ab concentration, and sample concentration. Overall two ELISAs were constructed and optimized for quantifying Smc1: phospho-Smc1 (pS957), and phospho-Smc1 (pS966).

Initially, production of recombinant phospho-Smc1 was pursued for use as a standard for the ELISAs. The Smc1 gene was inserted into an expression vector, and the sequence was verified. However, due to the known difficulties and expense associated with the expression and purification of recombinant proteins, synthetic phospho-peptide standards were generated as shown above in TABLE 4 and their efficacy was verified. As shown in TABLE 4 and FIG. 1, synthetic reference phospho-peptides were generated, each reference phosphopeptide comprising two independent epitopes corresponding to the sequence recognized by the ELISA capture antibody (the first epitope) and the sequence recognized by the ELISA detection antibodies (the second or third epitope).

For ELISAs employing the pS957 mAb (detection agent), the synthetic reference phosphopeptide (SEQ ID NO:4) included an N-terminus region comprising the second epitope (SEQ ID NO:2) from Smc1 (pS957) and a second C-terminus region comprising the first epitope (SEQ ID NO:1). The serine residue corresponding to S957 of the full length Smc1 polypeptide was synthesized using a phospho-serine amino acid.

Similarly, for ELISAs employing the pS966 MAb (detection agent), the synthetic reference phosphopeptide (SEQ ID NO:5) included a first N-terminus region comprising the third epitope (SEQ ID NO:3) from Smc1 (pS966) and a second C-terminus region comprising the first epitope (SEQ ID NO:1). The serine residue corresponding to 5966 of the full length Smc1 polypeptide synthesized using a phospho-serine amino acid.

As shown in TABLE 4 and FIG. 1, the first and second epitopes in the synthetic reference phosphopeptide (for binding to the capture and detection agents, respectively) are separated by a spacer region of 1 to about 50 amino acids to reduce the possibility of steric hindrance between the antibodies.

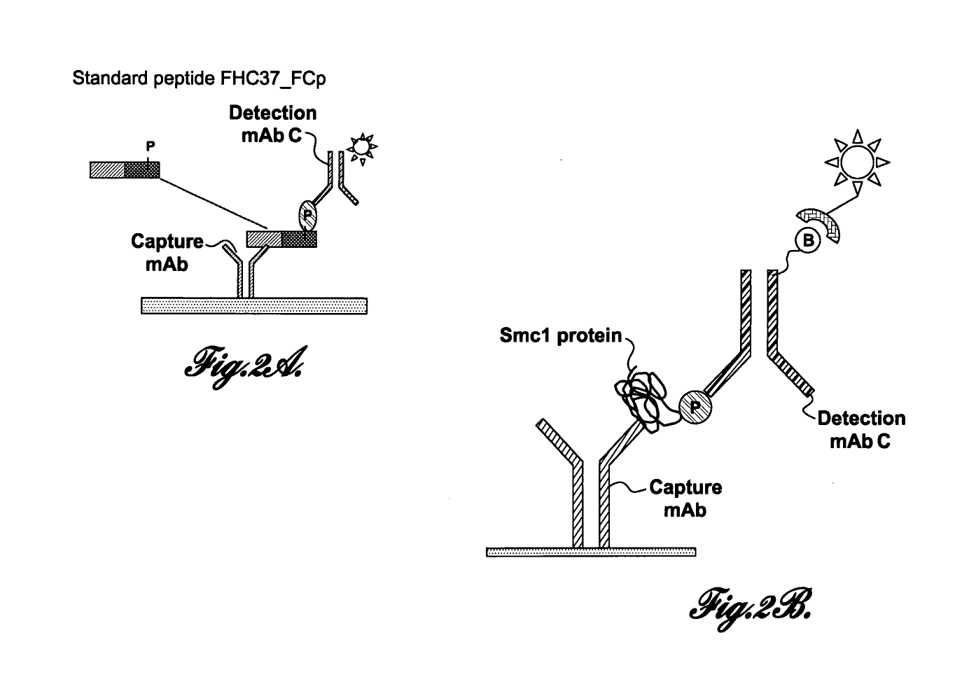

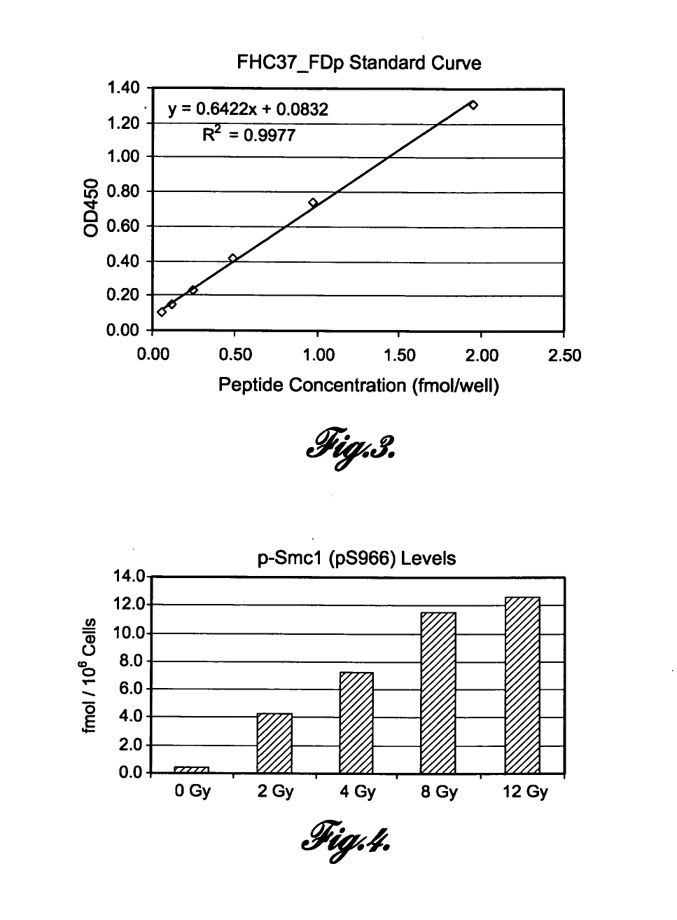

Reference Standard peptides were added to the ELISA plates and serially diluted two-fold to generate a seven point standard curve. The sample concentration is calculated by selecting the linear range of the standard curve (4 to 6 points) and deriving the equation of that line. As shown in FIG. 3, a standard curve was generated by plotting the concentration of the synthetic reference phosphopeptide (SEQ ID NO:5) versus OD450, a measure of the formation of a binding complex between the capture mAb, the synthetic reference phosphopeptide (SEQ ID NO:5), and the biotinylated detection mAb (e.g., as illustrated in FIG. 2B).

FIG. 4 graphically illustrates phospho-Smc1 (pS966) concentration in a Lymphoblast Cell Line (LBL) derived from the standard curve. An actively growing LBL was divided into five separate treatment flasks and either mock irradiated (OGy) or treated with the indicated dose of ionizing radiation (IR). Cells were harvested four hours after irradiation and protein lysates were prepared. Phospho-Smc1 (pS966) levels were determined by ELISA: The OD450 value for each lysate was converted to a molar concentration by way of the equation of the line generated with the standard reference peptide illustrated in FIG. 3.

As described above, the synthetic reference phosphopeptides were useful for generating a standard curve, which allows for the normalization of the amount of analyte protein within and between ELISA plates.

In summary, monoclonal antibodies to two phosphorylated forms of Smc1 were successfully generated. ELISAs were developed using the monoclonal antibodies and novel phospho-polypeptide standards. The ELISAs were validated for the ability to detect the time- and dose-dependent phosphorylation of Smc-1 in human LBL cells.

While the preferred embodiment of the invention has been illustrated and described, it will be appreciated that various changes can be made therein without departing from the spirit and scope of the invention.

1. A kit for detecting the presence or amount of a modified target protein within a sample, the kit comprising:
(i) a capture reagent that specifically binds to a first epitope on the target protein;
(ii) at least one detection reagent that specifically binds to a second epitope on the target protein; and
(iii) at least one synthetic hybrid reference peptide comprising the first epitope and the second epitope from the target protein of interest, wherein the synthetic reference peptide is capable of simultaneously binding to both the capture reagent and the at least one detection reagent, and wherein at least one of the first and second epitopes comprises a modified amino acid residue.

2. The kit of claim 1, wherein at least one of the capture reagent or the detection reagent is a polyclonal antibody, a monoclonal antibody or a fragment thereof.

3. The kit of claim 1, wherein the modification is selected from the group consisting of acetylation, amidation, deamidation, prenylation, formylation, glycosylation, hydroxylation, methylation, myristoylation, phosphorylation, ubiquitination, ribosylation or sulphation.

4. The kit of claim 3, wherein the modification is phosphorylation on tyrosine, serine, or threonine.

5. The kit of claim 4, wherein the synthetic hybrid reference peptide is a phosphopeptide comprising a phosphorylated amino acid at a modification site of interest.

6. The kit of claim 1, wherein the synthetic hybrid reference peptide further comprises an amino acid spacer region from 1 to about 50 amino acid residues between the first and second epitopes.

7. The kit of claim 2, wherein the detection reagent is an antibody or fragment thereof specific for the second epitope comprising a phosphorylated tyrosine, phosphorylated serine, or phosphorylated threonine.

8. The kit of claim 1, wherein the detection reagent is labeled by a detectable moiety selected from the group consisting of an enzyme, a fluorescent label, a stainable dye, a chemiluminescent compound, a colloidal particle, a radioactive isotope, a near-infrared dye, a DNA dendrimer, a water-soluble quantum dot, a latex bead, a selenium particle, and a europium nanoparticle.

9. The kit of claim 1, wherein the first epitope is an amino acid sequence comprising from 5 to about 200 amino acid residues of the target protein of interest, wherein the first epitope is derived from a separate location in the target protein from the second epitope.

10. The kit of claim 1, wherein the second epitope is an amino acid sequence comprising from 5 to about 200 amino acid residues of the target protein of interest.

11. The kit of claim 1, wherein the second epitope in the synthetic reference peptide consists of an amino acid sequence comprising from 5 to about 200 amino acid residues of the target protein of interest, said second epitope comprising at least one potential modification site.

12. The kit of claim 11, wherein the potential modification site is positioned such that at least 1 to 10 amino acid residues separate the first epitope from the potential modification site.

13. The kit of claim 1, wherein the kit comprises a first detection reagent specific for the second epitope comprising a phosphorylated tyrosine, phosphorylated serine, or phosphorylated threonine at a site of interest, and a second detection reagent specific for the second epitope comprising an unphosphorylated tyrosine, phosphorylated serine, or phosphorylated threonine at the site of interest.

14. The kit of claim 1, further comprising at least one detection reagent that specifically binds to a third epitope of the target protein, wherein the at least one synthetic hybrid reference peptide comprises the first epitope, the second epitope, and the third epitope from the target protein, and wherein the at least one of the first, second, and third epitopes comprises a modified amino acid.

15. The kit of claim 14, wherein the synthetic hybrid reference peptide further comprises at least one amino acid spacer region from 1 to about 50 amino acid residues disposed between two of the first, second, or third epitopes.

16. A method for generating an assay for reliably detecting the presence or amount of a modified target protein within a sample, the method comprising:
(i) generating a capture reagent that specifically binds to a peptide corresponding to a first epitope on a target protein;
(ii) generating at least one detection reagent that specifically binds to a peptide corresponding to a second epitope on the target protein;
(iii) generating at least one synthetic hybrid reference peptide comprising the amino acid sequence of the first epitope and the amino acid sequence of the second epitope from the target protein of interest, wherein the synthetic reference peptide is capable of simultaneously binding to both the capture reagent and the at least one detection reagent, and wherein at least one of the first epitope and second epitope comprises a modified amino acid residue; and
(iv) using the synthetic hybrid reference peptide as a quantitative standard for either (1) generating a standard curve for the assay, or (2) as a control in the assay.

17. The method of claim 16, further comprising contacting a sample with the capture agent and the at least one detection agent and detecting the presence or absence of the protein.

18. The method of claim 16, wherein at least one of the capture reagent or the detection reagent is a polyclonal antibody, a monoclonal antibody or fragment thereof.

19. The method of claim 16, wherein the modification is selected from the group consisting of acetylation, amidation, deamidation, prenylation, formylation, glycosylation, hydroxylation, methylation, myristoylation, phosphorylation, ubiquitination, ribosylation, or sulphation.

20. The method of claim 19, wherein the modification is phosphorylation on tyrosine, serine, or threonine.

21. The method of claim 19, wherein the synthetic reference peptide is a phosphopeptide comprising a phosphorylated amino acid at the modification site of interest.

22. The method of claim 16, wherein the synthetic hybrid reference peptide further comprises an amino acid spacer region from 1 to about 50 amino acid residues between the first and second epitopes.

23. The method of claim 20, wherein the detection reagent is an antibody or fragment thereof specific for the second epitope comprising a phosphorylated tyrosine, phosphorylated serine, or phosphorylated threonine.

24. The method of claim 16, wherein the detection reagent is labeled by a detectable moiety selected from the group consisting of an enzyme, a fluorescent label, a stainable dye, a chemiluminescent compound, a colloidal particle, a radioactive isotope, a near-infrared dye, a DNA dendrimer, a water-soluble quantum dot, a latex bead, a selenium particle, and a europium nanoparticle.

25. The method of claim 16, wherein the first epitope is an amino acid sequence comprising from 5 to about 150 amino acid residues of the target protein of interest, wherein the first epitope is derived from a separate location in the target protein from the second epitope.

26. The method of claim 16, wherein the second epitope is an amino acid sequence comprising from 5 to about 150 amino acid residues of the target protein of interest, said second epitope comprising at least one potential modification site.

27. The method of claim 16, wherein the second epitope in the synthetic hybrid reference peptide consists of an amino acid sequence comprising from 5 to about 25 amino acid residues of the target protein of interest, said second epitope comprising at least one potential modification site.

28. The method of claim 16, wherein the potential modification site is positioned such that at least 1 or more amino acid residues separate the first epitope from the potential modification site.

29. The method of claim 16, further comprising generating at least one detection reagent that specifically binds to a third epitope of the target protein, wherein the at least one synthetic hybrid reference peptide comprises the first epitope, the second epitope, and the third epitope from the target protein, and wherein the at least one of the first, second, and third epitopes comprises a modified amino acid.

30. The method of claim 29, wherein the synthetic hybrid reference peptide further comprises at least one acid spacer region from 1 to about 50 amino acid residues disposed between two of the first, second, or third epitopes.


SEQUENCE LISTING (14 sequences)

SEQ ID NO: 1 - protein, 15 aa, Artificial sequence
  Synthetic
DLTKYPDANPNPNEQ

SEQ ID NO: 2 - protein, 15 aa, Artificial sequence
  Synthetic
SQEEGSSQGEDSVSG

SEQ ID NO: 3 - protein, 11 aa, Artificial sequence
  Synthetic
DSVSGSQRISS

SEQ ID NO: 4 - protein, 28 aa, Artificial sequence
  Synthetic
ISQEEGSSQGEDSDLTKYPDANPNPNEQ

SEQ ID NO: 5 - protein, 28 aa, Artificial sequence
  Synthetic
EDSVSGSQRISSIDLTKYPDANPNPNEQ

SEQ ID NO: 6 - protein, 1233 aa, Homo sapiens
MGFLKLIEIENFKSYKGRQIIGPFQRFTAIIGPNGSGKSNLMDAISFVLGEKTSNLRVKTLRDLIHGAPVGKPAANRAFVSMVYSEEGAEDRTFARVIVGGSSEYKINNKVVQLHEYSEELEKLGILIKARNFLVFQGAVESIAMKNPKERTALFEEISRSGELAQEYDKRKKEMVKAEEDTQFNYHRKKNIAAERKEAKQEKEEADRYQRLKDEVVRAQVQLQLFKLYHNEVEIEKLNKELASKNKEIEKDKKRMDKVEDELKEKKKELGKMMREQQQIEKEIKEKDSELNQKRPQYIKAKENTSHKIKKLEAAKKSLQNAQKHYKKRKGDMDELEKEMLSVEKARQEFEERMEEESQSQGRDLTLEENQVKKYHRLKEEASKRAATLAQELEKFNRDQKADQDRLDLEERKKVETEAKIKQKLREIEENQKRIEKLEEYITTSKQSLEEQKKLEGELTEEVEMAKRRIDEINKELNQVMEQLGDARIDRQESSRQQRKAEIMESIKRLYPGSVYGRLIDLCQPTQKKYQIAVTKVLGKNMDAIIVDSEKTGRDCIQYIKEQRGEPETFLPLDYLEVKPTDEKLRELKGAKLVIDVIRYEPPHIKKALQYACGNALVCDNVEDARRIAFGGHQRHKTVALDGTLFQKSGVISGGASDLKAKARRWDEKAVDKLKEKKERLTEELKEQMKAKRKEAELRQVQSQAHGLQMRLKYSQSDLEQTKTRHLALNLQEKSKLESELANFGPRINDIKRIIQSREREMKDLKEKMNQVEDEVFEEFCREIGVRNIREFEEEKVKRQNEIAKKRLEFENQKTRLGIQLDFEKNQLKEDQDKVHMWEQTVKKDENEIEKLKKEEQRHMKIIDETMAQLQDLKNQHLAKKSEVNDKNHEMEEIRKKLGGANKEMTHLQKEVTAIETKLEQKRSDRHNLLQACKMQDIKLPLSKGTMDDISQEEGSSQGEDSVSGSQRISSIYAREALIEIDYGDLCEDLKDAQAEEEIKQEMNTLQQKLNEQQSVLQRIAAPNMKAMEKLESVRDKFQETSDEFEAARKRAKKAKQAFEQIKKERFDRFNACFESVATNIDEIYKALSRNSSAQAFLGPENPEEPYLDGINYNCVAPGKRFRPMDNLSGGEKTVAALALLFAIHSYKPAPFFVLDEIDAALDNTNIGKVANYIKEQSTCNFQAIVISLKEEFYTKAESLIGVYPEQGDCVISKVLTFDLTKYPDANPNPNEQ

SEQ ID NO: 7 - protein, 12 aa, Artificial sequence
  Synthetic
SQEEGSSQGEES

SEQ ID NO: 8 - protein, 12 aa, Artificial sequence
  Synthetic
SQEEGGSQGEES

SEQ ID NO: 9 - protein, 11 aa, Artificial sequence
  Synthetic
DSVSGSQRTSS

SEQ ID NO: 10 - protein, 11 aa, Artificial sequence
  Synthetic
ESVSGSQRTSS

SEQ ID NO: 11 - protein, 16 aa, Artificial sequence
  Synthetic
CGSGSQEEGSSQGEDS

SEQ ID NO: 12 - protein, 15 aa, Artificial sequence
  Synthetic
CGSGDSVSGSQRISS

SEQ ID NO: 13 - protein, 4 aa, Artificial sequence
  Synthetic
CGSG

SEQ ID NO: 14 - protein, 12 aa, Artificial sequence
  Synthetic
SQEEGSSQGEDS